(19) United States
(12) Patent Application Publication
(10) Pub. No.: US 2007/0049886 A1
(43) Pub. Date: Mar. 1, 2007

(54) Absorbent web with improved integrity and methods for making the same

(75) Inventor: Richard D. Mosbacher, Neenah, WI (US)

Correspondence Address: DORITY & MANNING, P.A., POST OFFICE BOX 1449, GREENVILLE, SC, 29602-1449, US

(73) Assignee: Kimberly-Clark Worldwide, Inc.

(21) Appl. No.: 11/218,929

(22) Filed: Aug. 31, 2005

(51) Int. Cl. A61F 13/15

(52) U.S. Cl. 604/365; 604/368; 604/380

(57) ABSTRACT

An absorbent web having an adhesive applied to at least one surface of the web in a manner such that the adhesive penetrates at least a portion of the thickness of the web is generally disclosed. The adhesive can bond the fibers, and other material, of the absorbent web to increase the integrity of the web. Also, a process for penetrating an adhesive into the thickness of an absorbent web is generally disclosed.

### BACKGROUND OF THE INVENTION

Absorbent articles and structures, such as absorbent pads, absorbent cores, and absorbent webs, have been formed by employing various techniques. The absorbent articles are typically incorporated into various absorbent products, such as diapers, feminine napkins and incontinence garments. In many applications, the absorbent articles are formed in an airlaying process.

Conventional airlaying processes typically include one or more forming chambers that are placed over a moving foraminous forming surface, such as a forming screen. Fibrous materials and/or particulate materials are introduced into the forming chamber and a vacuum source is employed to draw an airstream through the forming surface. The airstream deposits the fibers and/or particulate material onto the moving forming surface.

Once deposited onto the forming surface, a non-woven web is formed. Subsequently, the non-woven web can be bonded together. For example, the web can be bonded together using an adhesive and/or can be thermally bonded together.

In the past, flat non-woven sheets have been produced generally according to the above-described process. In other embodiments, the non-woven article has been shaped into a contoured batt or pad which locates more absorbent material or fibers in those areas which are subjected to higher levels of fluid loading.

In general depending upon their use, the airlaid articles should have good integrity and strength without significantly sacrificing the absorbency properties and/or wicking properties. For example, when used as an absorbent layer in a product, the airlaid web should quickly absorb fluids and, once absorbed, retain the fluids within the structure, without significantly changing shape.

### SUMMARY OF THE INVENTION

In one embodiment, the present disclosure is generally directed to an absorbent structure comprising an airlaid web and an adhesive. The airlaid web comprises natural fibers, synthetic fibers, or mixtures thereof. The airlaid web defines a first surface, a second surface, and a thickness measured from the first surface to the second surface. The adhesive is applied to the first surface of the airlaid web and penetrates at least about 25% of the thickness of the airlaid web. For example, the adhesive can penetrate at least about 50% of the thickness of the airlaid web, such as at least about 75% of the thickness of the airlaid web.

In one embodiment, at least a portion of the adhesive can penetrate the entire thickness of the airlaid web. For example, at least about 10% of the applied adhesive can penetrate the thickness of the airlaid web and exit the second surface of the airlaid web. When the adhesive penetrates the entire thickness of web, the adhesive can bond the second surface of the airlaid web to a second non-woven web.

In one embodiment, the airlaid web can also comprise superabsorbent materials. For example, the non-woven web can comprise at least about 50% superabsorbent materials by weight, such as at least about 60% superabsorbent materials by weight.

The adhesive can be any adhesive capable of penetrating the airlaid web. For example, in one embodiment, the adhesive can be a non-aqueous adhesive. Also, in one embodiment, the adhesive can have a viscosity of less than about 1500 centipoise.

In some embodiments, the absorbent structure can be utilized as an absorbent core of a personal care article for absorbing bodily fluids. For instance, the personal care article can be a diaper, a training pant, a feminine care product, an adult incontinence product, and the like.

In another embodiment, the present disclosure is generally directed to a process for penetrating an adhesive below a surface of an absorbent non-woven web. The process comprises providing a non-woven absorbent web, positioning the absorbent web in close proximity to at least one jet head, and applying an adhesive. The non-woven absorbent web can comprise natural fibers, synthetic fibers, or mixtures thereof. The absorbent web can define a first surface, a second surface, and a thickness measured from the first surface to the second surface. The absorbent web can be positioned in close proximity to at least one jet head. The jet head can have an orifice with a diameter of from about 0.003 to about 0.030. The adhesive can be applied to the first surface of the absorbent web through the jet head orifice at a pressure sufficient to penetrate the adhesive through at least about 25% of the thickness of the absorbent web. For example, the adhesive can be applied through the jet head orifice at a pressure of greater than about 1000 PSI, such as greater than about 1500 PSI. In one embodiment, the adhesive can be applied at a pressure of greater than about 2000 PSI.

In yet another embodiment, the present disclosure is generally directed toward a method of improving the integrity of an absorbent, airlaid web. The method comprises providing an airlaid web comprising a superabsorbent material and fibers and bonding a portion of the fibers and superabsorbent material in the web. The web can define a first surface, a second surface, and a thickness measured from the first surface to the second surface. The fibers can be selected from the group consisting of natural fibers, synthetic fibers, and mixtures thereof. The bonding can be achieved by applying a non-aqueous adhesive to the first surface of the airlaid web at a pressure sufficient to penetrate the adhesive into at least about 25% of the thickness of the airlaid web.

Various features and aspects of the present invention will be made apparent from the following detailed description.

### BRIEF DESCRIPTION OF THE FIGURES

A full and enabling disclosure of the present invention, including the best mode thereof to one of ordinary skill in the art, is set forth more particularly in the specification, which includes reference to the accompanying figures in which:

FIG. 1 is an illustration of one exemplary embodiment of an unfolded diaper having an absorbent core made of a non-woven absorbent web with penetrating adhesive lines;

FIG. 2 is an illustration of an exemplary embodiment of a non-woven absorbent web having penetrating adhesive lines;

FIG. 3 is an illustration of an exemplary apparatus and method of penetrating an adhesive into a non-woven absorbent web; and

FIG. 4 is a perspective view of a non-woven absorbent web having penetrating adhesive lines.

Repeated use of reference characters in the present specification and drawings is intended to represent the same or analogous features or elements of the present invention.

### DETAILED DESCRIPTION OF THE INVENTION

Reference now will be made to the embodiments of the invention, one or more examples of which are set forth below. Each example is provided by way of an explanation of the invention, not as a limitation of the invention. In fact, it will be apparent to those skilled in the art that various modifications and variations can be made in the invention without departing from the scope or spirit of the invention. For instance, features illustrated or described as one embodiment can be used on another embodiment to yield still a further embodiment. Thus, it is intended that the present invention cover such modifications and variations as come within the scope of the appended claims and their equivalents. It is to be understood by one of ordinary skill in the art that the present discussion is a description of exemplary embodiments only, and is not intended as limiting the broader aspects of the present invention, which broader aspects are embodied exemplary constructions.

In general, the present disclosure is directed to a non-woven absorbent web with an adhesive present in at least a portion of the thickness of the web. For instance, an adhesive can be applied to one surface of the absorbent web in such a manner that the adhesive penetrates below the surface of the absorbent web. The penetrating adhesive can provide integrity to the non-woven web by bonding the fibers within the web and/or bonding the absorbent web to a second structure, such as another non-woven web.

In one embodiment, the non-woven absorbent web can be an airlaid web, which can be utilized as an absorbent core in personal care products, such as diapers, feminine care products, adult incontinence products, training pants, and the like. Absorbent airlaid webs used in personal care products need to maintain their integrity, especially when wet, throughout the use of the products. Without proper integrity, the airlaid web in the absorbent core can collect in the middle portion, such as in the crotch region, of the personal care product when worn by a user. This loss of integrity can be especially present when the absorbent core is saturated with fluid.

However, applying an adhesive to the absorbent web, such that the adhesive penetrates at least a portion of the web, can provide integrity to the web, without significantly affecting the overall properties of the web. Also, the adhesive can reduce the bulk of the web, which may be desired depending on the end use of the web.

Penetrating the absorbent web with an adhesive can be accomplished by any method. The term “penetrate”, as used herein, means that the adhesive is applied in a manner such that the adhesive is present in the absorbent web beyond the surface to which it is applied. For example, the adhesive can penetrate at least about 25% of the thickness of the non-woven web. In some embodiments, the adhesive can penetrate at least about 50% of the thickness of the web, such as at least about 75%, or at least about 90% of the thickness of the web.

In some embodiments, a portion of the adhesive can penetrate the entire thickness (100% of the thickness of the web). In this embodiment, a portion of the applied adhesive can penetrate the entire thickness of the web, leaving the rest of the adhesive present, or impregnated, within the web. As used herein, the term impregnated means that at least some of the applied adhesive is located within the thickness of the web.

The portion of adhesive penetrating the entire thickness of the web can adhere the web to another surface, such as another non-woven web. Alternatively, the portion of the adhesive that penetrates the entire thickness of the web can be removed as excess adhesive. For example, in some embodiments, at least about 1% of the adhesive can penetrate the entire thickness of the web, such as at least about 5%. In other embodiments, at least about 10% of the adhesive can penetrate the entire thickness of the web, such as at least about 20%. In other embodiments, about 3% to about 15% of the applied adhesive can penetrated the entire web.

The penetrating adhesive can be applied to the absorbent web in any shape or manner. For example, in one particular embodiment, at least one line of penetrating adhesive can be present in the absorbent web. The at least one line can be oriented in the cross-machine direction (CD) of the non-woven web, the machine direction (MD) of the non-woven web, or in another direction that is not parallel to either the CD or MD direction. The thickness of the web is generally the measurement of the web in the Z-direction, perpendicular to the MD and CD directions. By penetrating the adhesive into at least a portion of the thickness of the absorbent web in a line, a “sewn affect” can be provided in the thickness of the web, giving integrity to the absorbent web in the direction of the adhesive line.

For example, referring to the exemplary embodiment of FIGS. 2 and 4, absorbent web 10 is shown having four penetrating adhesive lines 12. The adhesive penetrates at least a portion of the web's thickness, which is measured from first surface 11 to the second surface 13. However, in other embodiments, any number of adhesive lines 12 can be present in non-woven web 10. For example, in some embodiments, at least one line of penetrating adhesive can be present in non-woven web 10, such as at least about four adhesive lines 12 or at least about 6 adhesive lines 12. For instance, at least about ten penetrating adhesive lines 12 can be present in non-woven web 10, such as at least about thirty adhesive lines 12.

In embodiments having multiple adhesive lines 12, the lines 12 can be equally spaced, such as shown in FIGS. 2 and 4. However, in other embodiments, the adhesive lines 12 can be arranged according to any pattern or spacing.

The adhesive lines 12 shown in FIGS. 2 and 4 are continuous lines. However, in other embodiments, the adhesive lines 12 can be discontinuous lines. In yet other embodiments, the penetrating adhesive can be in other shapes, such as dots, circles, designs, patterns, or the like. Any design or pattern of penetrating adhesive can be utilized in accordance with the present disclosure.

Furthermore, penetrating adhesive lines 12 can vary in the amount of penetration throughout each line. For example, each line can have a different amount of penetration into the thickness of the web than the other lines present in the web. Also, each line can vary in the amount of penetration throughout the line itself. For example, each line can have different levels of adhesive penetration through the line.

The penetrating adhesive can be applied to a relatively small percentage of the overall surface area of the absorbent web, while remaining effective to provide integrity to the web. For example, less than about 15% of the surface area of the web can be penetrated with an adhesive, such as from about 1% to about 10%. For instance, in one embodiment, the penetrating adhesive can be applied to less than about 10% of the surface area of the web, such as from about 0.5% to about 5%.

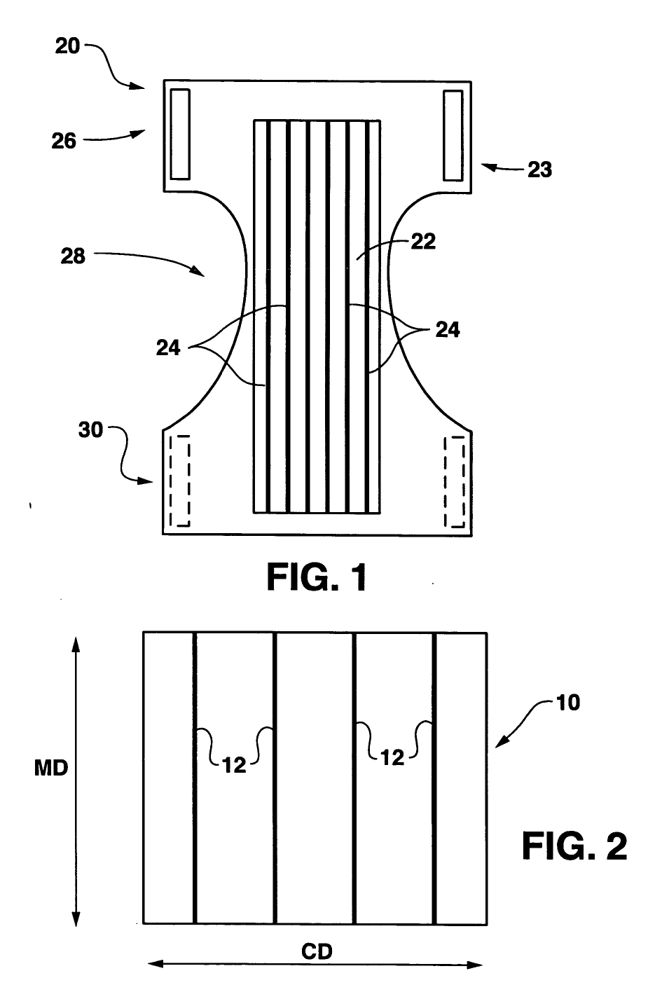

The absorbent web with penetrating adhesive present can be utilized in any personal care product, such as diapers, disposable training pants, feminine care products, adult incontinence products, and the like. Referring to the FIG. 1 as an exemplary embodiment, a diaper 20 is shown having an absorbent core 22. The structure of diapers is well known in the art. FIG. 1 represents a typical diaper where the absorbent core 22 is located between a liquid impermeable outer layer 23 and a liquid permeable, skin contacting, inner layer (not shown).

Although a diaper is described as an example of a personal care product, it is understood that the present disclosure is equally applicable to other absorbent products intended to absorb body fluids. Examples of personal care products that can incorporate the absorbent web with penetrating adhesive of the present disclosure, such as in the absorbent core, are described in U.S. Pat. No. 6,867,343 to La Fortune, U.S. Pat. No. 6,793,650 to Weber, U.S. Pat. No. 6,710,225 to Everett, et al., U.S. Pat. No. 6,783,487 to Duhm, et al., and U.S. Pat. No. 6,656,168, to Braverman, et al. all of which are herein incorporated by reference.

The absorbent core 22 can be, for example, a non-woven absorbent web, such as an airlaid non-woven web. FIG. 1 shows absorbent core 22 having six penetrating adhesive lines 24. However, in other embodiments, any number of penetrating adhesive lines 24 can be present in absorbent core 22. For example, in some embodiments, at least one penetrating adhesive line 24 is present in absorbent core 22, such as at least four adhesive lines or at least six adhesive lines 24. For instance, at least ten adhesive lines 24 can be present in non-woven web 10, such as at least thirty adhesive lines 24.

Penetrating adhesive lines 24 are shown to be oriented in a direction running from the front portion 26 through middle portion 28 into back portion 30, which is the machine direction (MD) of the absorbent core 22. However, penetrating adhesive lines 24 can be oriented in any direction, such as in the cross-machine direction (CD) of the absorbent core 22. Also, the penetrating adhesive lines 24 can only extend a portion of the length of the absorbent core 22. For example, in one embodiment, adhesive lines 24 could be located only on the absorbent core in the back portion 30. Alternatively, or in addition to, penetrating adhesive lines 24 can be present only on the front portion 26 of diaper 20. For example, locating the adhesive lines 24 in the front portion 26 and/or the back portion 30 can help prevent the absorbent core 22 from clumping toward the middle portion 28 when worn by the user. In fact, any arrangement of the penetrating adhesive on absorbent core 22 is within the scope of this disclosure.

As shown, absorbent core 22 is rectangular in shape. However, absorbent core 22 can have any of a number of shapes. For example and without limitation, absorbent core 22 can be rectangular, I-shaped or T-shaped. In such products as e.g. diapers, pants, and the like, absorbent core 22 is preferably narrower in the middle portion 28 than in the front portion 26 or the rear portion 30, especially where the middle portion 28 of the absorbent article is narrower than the rear portion 30 or the front portion 26.

When the absorbent web with penetrating adhesive present is used as an absorbent core in a personal care article, the absorbent web can be any absorbent non-woven web, such as an airlaid non-woven web. For example, the airlaid web can comprise fluff fibers and/or superabsorbent materials.

The at least one line of penetrating adhesive can provide integrity to the absorbent core, even when the absorbent core is saturated. Also, if desired, the penetrating adhesive can penetrate the entire thickness of the absorbent core to adhere the absorbent core to another layer of the personal care article. For example, the penetrating adhesive lines as shown in FIG. 1 can penetrate the entire thickness of the absorbent core 22 to adhere the absorbent core 22 to the liquid impermeable outer layer 23. Alternatively, or in addition, the penetrating adhesive can adhere the absorbent core 22 to the liquid permeable inner layer of the diaper.

Any process can be used to provide the at least one line of penetrating adhesive within the absorbent web. For example, in one particular embodiment, modified hydro-entangling equipment can be utilized to penetrate an adhesive into an absorbent web. Hydro-entangling equipment is well known to one of ordinary skill in the art. For example, typical hydro entangling equipment and processes are described in U.S. Pat. No. 5,284,703 issued to Everhart, et al. and U.S. Pat. No. 5,137,600 issued to Barnes, et al., both of which are incorporated by reference herein. The hydro-entangling method and apparatus must be modified for use with an adhesive, such as a glue, instead of the fluid, which is typically water.

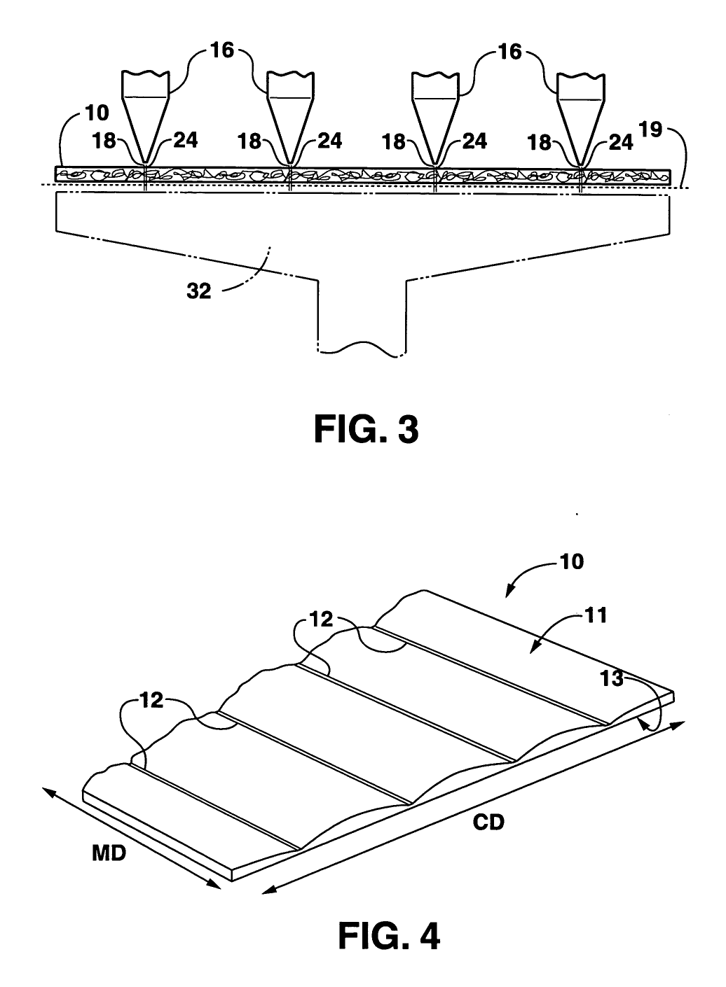

For example, referring to the exemplary embodiment of FIG. 3, penetrating the adhesive 14 into absorbent web 10 can be achieved by passing the absorbent web 10 under one or more jet heads 16. The jet heads 16 of adhesive 14 can drive the adhesive 14 into, and possibly through, the thickness of the absorbent web 10 to form the adhesively penetrated absorbent web. In order to apply the penetrating adhesive into the thickness of the web, the web can be positioned in close proximity to the jet heads 16. For example, the

In this exemplary penetrating process, the adhesive 14 passes through at least one hole or orifice 18 located on the tip 19 of jet head 16. Any number of holes or orifices 18 may be present on the tip 19 of jet head 16, such as at least one hole or orifice 18. However, typically each jet head 16 has only one hole or orifice 18. In one embodiment, a manifold (not shown) can contain all or a portion of jet heads 16. The manifold can evenly distribute the adhesive 14 to each jet head 16. Also, the number of jet heads 16 on the manifold can dictate how many penetrating adhesive lines are present on the absorbent web 10. For example, in one embodiment, at least four jet heads 16 are present on the manifold. In other embodiments, at least six orifices are present on the manifold. For example, a hydro-entangling manifold produced by Honeycomb Systems, Inc. of Biddeford, Me., having a strip of 0.007 inch diameter jet heads, each with one orifice, thirty orifices per inch, and one row of orifices can be used according to the present disclosure. However, many other manifold configurations and combinations may be used. Also, a single manifold may be used or several manifolds may be arranged in succession.

In the penetrating process, the adhesive 14 passes through each jet head 16 and orifice 18 at a sufficient pressure to allow the adhesive to penetrate the thickness of the non-woven web the desired amount. The pressure needed to penetrate the adhesive into the thickness of the web can vary according to several different factors, including, but not limited to, the type of adhesive, the viscosity of the adhesive, the size of the orifice, the desired depth of penetration of the adhesive, and the type of non-woven web. However, typical pressures that can be used to pass the adhesive 14 through the orifice 18 can be greater than about 1,000 lbs. per square inch (psi), such as greater than about 1,200 psi. For example, the adhesive 14 can pass through the orifice 18 at pressures of greater than about 1,500 psi, such as greater than about 2,000 psi.

The size of the orifice 18 can also vary on several factors, including, but not limited to, the type of adhesive, the viscosity of the adhesive, the pressure at which the adhesive passes through the orifice, the desired depth for the adhesive to penetrate, the type of non-woven web, and the desired size of the penetrating adhesive line. Typically, the orifices 18 may be from about 0.003 to about 0.03 inches in diameter. For example, in one embodiment, the orifices 18 can have a diameter of from about 0.007 to about 0.02 inches, such as about 0.008 to about 0.01 inches. When formed from jet heads 16, the width of adhesive lines 12 can typically be the size of the orifice 18 utilized to penetrate the absorbent web 10 with the adhesive 14, although some diffusion may occur below the surface of the web.

The use of jet heads 16 can provide continuous adhesive lines 12 in the non-woven web through a continuous flow of adhesive 14. However, other patterns of designs can be produced using jet heads 16. For example, discontinuous adhesive lines can be produced by pulsating the flow of adhesive 14 through jet heads 16.

The absorbent web 10 can be penetrated with adhesive 14 while the absorbent web 10 is still on its forming surface or after the non-woven web has been positioned onto another non-woven web. For example, as shown in FIG. 4, when penetrating the absorbent web 10 on a forming surface, the forming surface 19 can be a foraminous screen (i.e., mesh fabric) where the web was formed, such as through air laying or wet laying. As such, when the adhesive is applied to the absorbent web in a manner such that at least a portion of the adhesive penetrates the entire thickness of the web, any excess adhesive can be collected through the forming fabric and removed from the non-woven web. For example, referring to FIG. 4, a vacuum and/or collection apparatus 32 could be utilized to collect any excess adhesive 14 that penetrated the entire thickness of the absorbent web 10.

Alternatively, the absorbent web can be positioned on a second surface, such as a non-woven web, when the entire thickness absorbent web is penetrated with an adhesive. In this embodiment, the adhesive can be applied to the side opposite of the side that contacts the second surface. As such, any adhesive that penetrates the entire thickness of the absorbent web can adhere the opposite side of the absorbent web to the second surface. As such, the penetrating process can be utilized to form a laminate structure of two non-woven webs. For example, when the absorbent web is intended for use as an absorbent core in a personal care article, the penetrating process can adhere the absorbent core to another structure of the personal care product. For instance, the penetrating adhesive can adhere the absorbent core to the liquid impervious outer layer and/or the liquid pervious inner layer.

The absorbent web can be formed through any process and can include any fiber material that can be used to form the web. For instance, the web can include natural fibers, synthetic fibers, and combinations thereof. Examples of natural fibers include wood pulp fibers, cotton fibers, wool fibers, silk fibers, and the like, as well as combinations thereof. Wood pulp fibers can include softwood fibers and hardwood fibers. Synthetic fibers can include rayon fibers, polyolefin fibers, polyester fibers, and the like, as well as combinations thereof. Polyolefin fibers include polypropylene fibers and polyethylene fibers. The fibers can have various lengths, such as up to about six to about eight millimeters or greater.

In a particular embodiment, the absorbent non-woven web 10 can be an airlaid web. Airlaid webs can comprise fluff fibers. Air laying processes are well known in the art. For instance, airlaid webs and air laying processes can be those, disclosed in U.S. Publication No. 2002/0180092 A1 of Abba, et al. or U.S. Publication No. 2003/0036741 A1 also of Abba, et al., both of which are incorporated herein.

The absorbent webs can also include various additives. For example, additives such as odor absorbents, anti-microbial agents, scented materials, and the like can be incorporated into the web. In one particular embodiment, when the absorbent web is an airlaid web, the absorbent web can also include a matrix of hydrophilic fibers, such as a web of fluff fibers, mixed with particles of a high-absorbency material (commonly known as superabsorbent material). For example, the absorbent core can include a matrix of airlaid fluff fibers and superabsorbent hydrogel-forming particles. The fluff fibers can be exchanged with synthetic, polymeric, meltblown fibers or with a combination of meltblown fibers and natural fibers. The superabsorbent particles can be substantially homogeneously mixed with the hydrophilic fibers or can be nonuniformly mixed.

For example, when used as an absorbent core of a personal care product, the airlaid web fibers and superabsorbent particles can also be selectively placed into desired zones of the absorbent core to better contain and absorb body exudates. The concentration of the superabsorbent particles can be uniform or can vary through the thickness of the absorbent core. Alternatively, the absorbent core can include a laminate of fibrous webs and superabsorbent material or other suitable means of maintaining a superabsorbent material in a localized area.

In one particular embodiment, an airlaid web can contain a high amount of superabsorbent material relative to the fibers of the airlaid web. For example, in some embodiments, the absorbent airlaid web can be greater than about 50% superabsorbent material by weight, such as greater than about 60%. For instance, the non-woven absorbent core can be greater than about 65% superabsorbent material by weight, such as greater than about 70%. In one embodiment, the non-woven absorbent core can be greater than about 75% superabsorbent material by weight, such as greater than about 80%. Without wishing to be bound by theory, it is believed that these higher levels of superabsorbent material present in the absorbent web can be realized because of the added integrity given to the absorbent core from the penetrating adhesive.

Superabsorbent materials are produced in a wide variety of forms including, but not limited to, particles, fibers and flakes. Superabsorbent material can be a water-swellable, water-insoluble organic or inorganic material capable, under the most favorable conditions, of absorbing at least about 15 times its weight and, more desirably, at least about 30 times its weight in an aqueous solution containing 0.9 weight percent sodium chloride. The superabsorbent materials can be natural, synthetic and modified natural polymers and materials. In addition, the superabsorbent materials can be inorganic materials, such as silica gels, or organic compounds such as cross-linked polymers. Suitable superabsorbent materials can be selected from natural, synthetic, and modified natural polymers and materials. The superabsorbent materials can be inorganic materials, such as silica gels, or organic compounds, such as cross-linked polymers. Suitable superabsorbent materials are available from various commercial vendors, such as Dow Chemical Company located in Midland, Mich., U.S.A., and Stockhausen GmbH & Co. KG, D-47805 Krefeld, Federal Republic of Germany. Typically, a superabsorbent material (SAM) is capable of absorbing at least about 15 times its weight in water, and desirably is capable of absorbing more than about 25 times its weight in water.

A wide variety of superabsorbent materials are known to those skilled in the art. See, for example, U.S. Pat. No. 4,076,663 issued Feb. 28, 1978, to Masuda et al.; U.S. Pat. No. 4,286,082 issued Aug. 25, 1981, to Tsubakimoto et al.; U.S. Pat. No. 4,062,817 issued Dec. 13, 1977, to Westerman; and U.S. Pat. No. 4,340,706 issued Jul. 20, 1982, to Obayashi et al, all of which are incorporated by reference.

The basis weights of the absorbent webs can vary dramatically dependent upon the particular application of the non-woven web. For example, when a non-woven web is an airlaid web, the airlaid web can have a basis weight of at least about 40 GSM, and particularly from about 50 GSM to about 1500 GSM or higher. In one embodiment, the basis weight of the web can be from about 50 GSM to about 700 GSM.

The overall density of the absorbent web can also vary dependent upon the materials used to form the web, the process used to form the web, and the desired result. For example, when the web is an airlaid web, the density of the web can generally be from about 0.01 grams per cubic centimeter to about 0.3 grams per cubic centimeter.

Any adhesive that is capable of penetrating the web can be used in accordance with the present disclosure. For example, the adhesive can be any glue, hot melt adhesive, or the like. For example, in one embodiment, the adhesive must be capable of being injected through the small orifices, such as those described above, at high pressures to penetrate the absorbent web. In the embodiments where the non-woven web is an airlaid web containing superabsorbent material, a non-aqueous adhesive should be used, such as a latex adhesive, to avoid any interaction of the superabsorbent material and water.

Varying properties of the adhesive can help control the amount of adhesive penetration into the thickness of the web. For example, changing the viscosity of the adhesive can alter the amount of penetration into the web. Also, the viscosity of the adhesive can somewhat dictate the size of the orifice and the pressure. For example, a lower viscosity adhesive can penetrate further into the thickness of the absorbent web than a higher viscosity adhesive applied at the same pressure. For instance, in one embodiment, the adhesive can have a viscosity at application temperature of less than about 2000 centipoise, such as less than about 1500 centipoise. For example in some embodiments, the adhesive can have a viscosity at the application temperature of less than about 1000 centipoise.

The application temperature of the adhesive can vary according to the particular adhesive applied. For instance, in some embodiments, the application temperature can be at least about 300° F., such as from about 300° F. to about 450° F. Typically, varying the application temperature can alter the viscosity of the adhesive. For example, in some instances, increasing the application temperature can lower the viscosity, which could lead to greater penetration by the adhesive.

These and other modifications and variations to the present invention may be practiced by those of ordinary skill in the art, without departing from the spirit and scope of the present invention, which is more particularly set forth in the appended claims. In addition, it should be understood the aspects of the various embodiments may be interchanged both in whole or in part. Furthermore, those of ordinary skill in the art will appreciate that the foregoing description is by way of example only, and is not intended to limit the invention so further described in the appended claims.

What is claimed:

1. An absorbent structure, comprising:
an airlaid web comprising natural fibers, synthetic fibers, or mixtures thereof, said airlaid web defining a first surface, a second surface, and a thickness measured from said first surface to said second surface; and
an adhesive applied to said first surface of said airlaid web, said adhesive penetrating at least about 25% of the thickness of said airlaid web.

2. An absorbent structure as in claim 1, wherein said adhesive penetrates at least about 50% of the thickness of said airlaid web.

3. An absorbent structure as in claim 1, wherein at least a portion of said adhesive penetrates the entire thickness of said airlaid web.

4. An absorbent structure as in claim 3, wherein at least about 10% of said applied adhesive penetrates the entire thickness of said airlaid web and exits said second surface of said airlaid web.

5. An absorbent structure as in claim 3, wherein said adhesive bonds said second surface of said airlaid web to a second non-woven web.

6. An absorbent structure as in claim 1, wherein said airlaid comprises at least about 50% superabsorbent materials by weight.

7. An absorbent structure as in claim 1, wherein said adhesive is a non-aqueous adhesive.

8. An absorbent structure as in claim 1, wherein the absorbent structure is an absorbent core of a personal care article for absorbing bodily fluids selected from the group consisting of diapers, training pants, feminine care products, and adult incontinence products.

9. A process for penetrating an adhesive below a surface of an absorbent non-woven web, comprising:
providing an airlaid absorbent web comprising natural fibers, synthetic fibers, or mixtures thereof, wherein the absorbent web defines a first surface, a second surface, and a thickness measured from the first surface to the second surface; and
applying an adhesive to the first surface the absorbent web at a pressure sufficient to penetrate the adhesive through at least about 25% of the thickness of the absorbent web, the adhesive being applied through a jet head having an orifice, the office having a diameter of from about 0.003 inches to about 0.03 inches.

10. A process for penetrating an adhesive as in claim 9, wherein the adhesive is applied to the absorbent web through the jet head orifice at a pressure of greater than about 1000 PSI.

11. A process for penetrating an adhesive as in claim 9, wherein the adhesive penetrates at least about 50% of the thickness of the absorbent web.

12. A process for penetrating an adhesive as in claim 9, wherein a portion of the adhesive penetrates the entire thickness of the absorbent web.

13. A process for penetrating an adhesive as in claim 12, further comprising:
adhering the second surface of the absorbent web to a second non-woven web, wherein the portion of the adhesive that penetrates the entire thickness of the absorbent web adheres the second surface of the absorbent web to the second non-woven web.

14. A process for penetrating an adhesive as in claim 13, wherein the second non-woven web is an absorbent non-woven web.

15. A process for penetrating an adhesive as in claim 13, wherein the second non-woven web is liquid impermeable non-woven web.

16. A process for penetrating an adhesive as in claim 12, further comprising:
removing the portion of adhesive that penetrates the entire thickness of the non-woven web from the second surface of the absorbent web.

17. A process for penetrating an adhesive as in claim 9, wherein the airlaid web further comprises superabsorbent material.

18. A process for penetrating an adhesive as in claim 17, wherein the airlaid web comprises at least about 50% superabsorbent material by weight.

19. A process for penetrating an adhesive as in claim 9, wherein the adhesive has a viscosity of less than about 1500 centipoise.

20. A method of improving the integrity of an absorbent, airlaid web comprising:
providing an airlaid web defining a first surface, a second surface, and a thickness measured from the first surface to the second surface, wherein the airlaid web comprises a superabsorbent material and fibers selected from the group consisting of natural fibers, synthetic fibers, and mixtures thereof; and
bonding a portion of the fibers and superabsorbent material in the airlaid web by applying a non-aqueous adhesive to the first surface of the airlaid web at a pressure sufficient to penetrate the adhesive into at least about 25% of the thickness of the airlaid web.
